(19) United States
(12) Patent Application Publication
(10) Pub. No.: US 2023/0050418 A1
(43) Pub. Date: Feb. 16, 2023

(54) PRODUCTION OF BENZENE DERIVATIVES

(71) Applicants: RHODIA OPERATIONS, Lyon (FR); LE CENTRE NATIONAL DE LA RECHERCHE SCIENTIFIQUE, Paris (FR); UNIVERSITE DE POITIERS, Poitiers (FR)

(72) Inventors: Ivan Scodeller, San Vito Al Tagliamento (IT); Francois Jerome, Sevres-Anxaumont (FR); Karine De Oliveira Vigier, Fontaine Le Comte (FR); Changru Ma, Shanghai (CN); Raphael Johannes Wischert, Shanghai (CN); Eric Muller, Lyon (FR)

(73) Assignees: RHODIA OPERATIONS, Lyon (FR); LE CENTRE NATIONAL DE LA RECHERCHE SCIENTIFIQUE, Paris (FR); UNIVERSITE DE POITIERS, Poitiers (FR)

(21) Appl. No.: 17/784,884

(22) PCT Filed: Dec. 13, 2019
(86) PCT No.: PCT/CN2019/125046

§ 371 Date: Jun. 13, 2022

(51) Int. Cl. C07D 491/08

(52) U.S. Cl. CPC C07D 491/08

(57) ABSTRACT

The present invention relates to the production of benzene derivatives from furfural and its derivatives. The present invention also relates to the preparation of novel intermediates derived from furfural and its derivatives. The present invention describes new routes for converting furfural and its derivatives into benzene derivatives including novel intermediates.

The present invention relates to the production of benzene derivatives and in particular ortho-xylenediamine, meta-xylenediamine and 1,2,3-tri(aminomethyl)benzene from furfural and its derivatives. The invention describes new routes for converting furfural and its derivatives into benzene derivatives including novel intermediates.

In recent times, a tendency has grown to obtain a variety of chemicals from renewable sources. In this context, there has been a tendency to create chemicals from biomass carbohydrates, such as cellulose, starch, hemicellulose, sugars and the like. Under dehydration conditions, these carbohydrates can be converted into a number of interesting chemicals, including furfural, hydroxymethyl furfural and derivatives thereof. There is an interest to use these chemicals for the production of value-added chemical compounds. Examples of such value-added chemical compounds include orthophthalic acid (commonly named phthalic acid), terephthalic acid, isophthalic acid, trimellitic acid, and other benzene derivatives that contain two or more carboxylic moiety substituents.

One approach to transfer furan, furfural and their derivatives into chemically more valuable six-membered ring aromatic compounds is the Diels-Alder reaction between the furan ring system and ethylene or ethylene derivatives.

The Diels-Alder reaction with furan derivatives is known. The Diels-Alder reaction of furan and ethylene to 3,6-epoxycyclohexane has been described in U.S. Pat. No. 2,405,267:

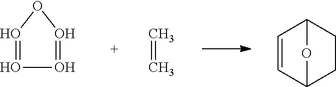

WO 2010/151346 describes the conversion of 2,5-dimethylfurane to para-xylene.

A process for the preparation of a substituted benzene derivative by reacting a furfuryl ether with an ethylene derivative is described in WO 2013/048248.

WO 2014/065657 broadly claims a process for the preparation of benzene derivatives by reacting a furan derivative with ethylene. The furan derivative may bear at 2 and 5 position a variety of substituents including alkyl, aralkyl, —CHO, —CH₂OR³, —CH(OR⁴)(OR⁵) and —COOR⁶. However, this document provides examples only with 2,5-dimethylfurane, 2-methylfurane, 2,5-furane dicarboxylic acid and the dimethylester of 2,5-furane dicarboxylic acid. In particular, there is no example wherein furfural is converted into a benzene derivative.

Yu-Ting Cheng, et al. in Green Chem., 2012, 14, 3314-3325 provide an overview over the production of targeted aromatics by using Diels-Alder classes of reactions with furans and olefins. The authors found that while furan, methylfuran and dimethylfuran react smoothly with olefins, the first step for furfural conversion is decarbonylation to form furan and CO. The produced furan then enters the known furan conversion reaction:

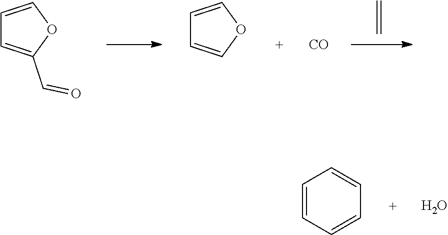

These difficulties in reacting furfural with olefins are confirmed in WO 2014/197195. Although the authors of this document conducted screening experiments, testing range of various solvents, catalysts, reaction temperatures, pressures and times, and 5-hydroxy-2-furfural concentrations, they failed to identify a system in which 4-hydroxymethylbenzaldehyde formed (example 4.3).

The authors suggest solving this problem by air oxidation of 5-hydroxymethyl-2-furfural to generate the corresponding 5-hydroxymethyl-2-furoic acid or other oxidized derivative, which has been shown to work well in the Diels-Alder reaction with olefins.

Both, decarbonylation of furfural to furan as well as oxidation of furfural to furoic acid has the disadvantage that the aldehyde substituent at the furan ring is lost. As a consequence thereof, the aldehyde substituent is no longer present in the obtained Diels-Alder adduct which makes it more difficult to obtain benzaldehyde derivatives from furan derivatives and in particular furfural. However, benzaldehyde derivatives are desirable as valuable intermediates in the preparation of other important chemical compounds, such as meta-xylenediamine, ortho-xylenediamine and 1,2,3-tri(aminomethyl)benzene.

In order to solve the above problems, various experiments were conducted in an effort to react furfural with ethylene derivatives. As expected from the prior art, no reaction between furfural and acrylonitrile was observed even under varying conditions with respect to catalyst, molar ratio of the reactants, temperature and reaction time. The aldehyde substituent of furfural was then converted into its diethyl-ketal. Ketals are known derivatives of aldehydes from which the desired aldehyde can easily be obtained by removing the alcohol. However, when reacting the diethyl-ketal of furfural with acrylonitrile, only traces of the Diels-Alder adduct, the oxanorbornene were observed. Upon further investigations, it was then found that cyclic ketals of furfural surprisingly react with acrylonitrile thereby forming the desired Diels-Alder adduct (WO 2017/097220).

The Diels-Alder reaction of furfural ethylene acetal with maleic anhydride is described by S. Takano in Yakugaku Zasshi 102 (2) 153-161 (1982).

In the above described reaction between the cyclic ketal of furfural with acrylonitrile the product can be the ortho or meta isomer of the Diels-Alder

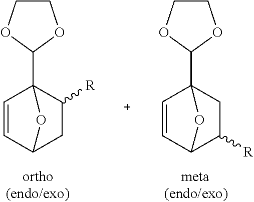

adduct:

Further investigations revealed that if acrylonitrile is used as dienophile, the meta/ortho ratio in the obtained Diels-Alder adduct remains close to 1, independent of reaction temperature and catalyst. For the production of certain benzene and in particular xylene derivatives it would, however, be desirable if the ortho or meta isomer of the Diels-Alder adduct would be obtained at higher ratio. In particular, for the production of meta-xylene derivatives it would be advantageous if the meta/ortho ratio of the Diels-Alder adduct could be increased.

The present inventors have now found that the meta/ortho ratio in the Diels-Alder adduct can surprisingly be increased if the cyclic ketal of furfural is reacted with a dienophile comprising an acryloyl group instead of the acrylonitrile used in the prior art.

The present invention therefore relates to a process for the preparation of a compound of Formula (I)

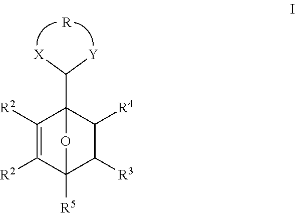

wherein
X and Y independently are optionally substituted heteroatoms;
R is a C₁₋₄ alkylene group which may optionally be substituted with one or more R¹;
R¹ is a linear, branched and/or cyclic, saturated or unsaturated hydrocarbon group which optionally bears one or more functional groups;
R² independently is H, alkyl, alkenyl or aryl;
R³ and R⁴ independently are H, —COR² or —CO₂R², provided that R³ and R⁴ are not identical;
R⁵ is R², —CH₂OR², —COR², —CO₂R² or

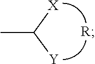

which comprises reacting a compound of the Formula (II)

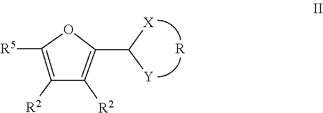

wherein X, Y, R, R² and R⁵ are defined as above;
with a compound of the Formula (III) or (III′)

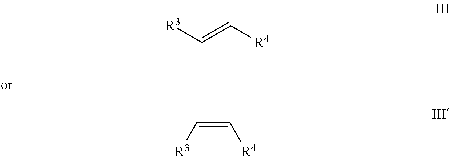

wherein R³ and R⁴ are defined as above.

The furan derivative of Formula (II) that is being used as starting material can be derived from a biomass resource. For example, the furan derivative can be derived from the dehydration of a carbohydrate. The carbohydrate is suitably selected from polysaccharides, oligosaccharides, disaccharides and monosaccharides. Suitable biomass sources as well as suitable methods for their conversion into furfural derivatives are known to the person skilled in the art. Alternatively, the furfural derivative can be a commercially available chemical product obtained by usual chemical reactions.

In the furfural derivative of Formula (II) the aldehyde residue of furfural is present as cyclic ketal. However, the present invention is not limited to furfural and its cyclic ketal derivative but also includes furan derivatives comprising heteroatoms other than O. Therefore, X and Y in the compound of Formula (II) are independently of each other optionally substituted heteroatoms, such as O, S and N. In this context, “optionally substituted” defines that the heteroatom may bear a substituent, if required. If the heteroatom cannot bear any further substituent, no substituent is present. For example, if the heteroatom is O or S, there is no substituent at the heteroatom. However, if the heteroatom is N, then X and Y may be —NH— or —N(substituent)—. This substituent has the same meaning as R¹. Thus, X and Y are preferably independently selected from —O—, —S—, —NH—, and —N(R¹)—, more preferably from —O— and —S—. Most preferably, X and Y are both O or both S.

In the furfural derivative of Formula (II), R is a C₁₋₄ alkylene group, preferably a C₂₋₄ alkylene group, more preferably, a C₂₋₃ alkylene group, most preferably a C₂ alkylene group. This alkylene group may optionally be substituted with one or more R¹ substituents. R¹ is a linear, branched and/or cyclic, saturated or unsaturated hydrocarbon group which optionally bears one or more functional groups. Such hydrocarbon groups include all chemical moieties comprising carbon atoms, preferably from 1 to 24 carbon atoms besides the required number of hydrogen atoms. Examples of linear, branched, and/or cyclic, saturated or unsaturated hydrocarbon groups are alkyl, alkenyl, alkynyl, aromatic groups, etc. The hydrocarbon group may optionally bear one or more functional groups which means that the hydrocarbon group may contain one or more heteroatoms, such as O, N and S, or functional groups, such as —CO— or —COO—. Furthermore, the hydrocarbon group may be substituted with functional groups, such as nitro, nitroso, sulfo, sulfonate, cyano, cyanato, thiocyanato, amino, hydroxyl, carboxyl, etc.

Representative examples of R¹ will now be explained in more detail, thereby also providing definitions of certain terms which are applicable throughout the present specification and in particular also for all other substituents, if not defined otherwise.

The term “alkyl” as used herein refers to a linear, branched, or cyclic saturated hydrocarbon group typically although not necessarily containing 1 to about 24 carbon atoms, preferably 1 to about 12 carbon atoms, or 1 to about 6 carbon atoms, 1 to about 3 carbon atoms. Certain embodiments provide that the alkyl is methyl, ethyl, n-propyl, isopropyl, n-butyl, isobutyl, tert-butyl, octyl, decyl, or the like, as well as cycloalkyl groups such as cyclopentyl, cyclohexyl or the like. Generally, although again not necessarily, alkyl groups herein contain 1 to about 12 carbon atoms. The term “lower alkyl” intends an alkyl group of 1 to 6 carbon atoms, and the specific term “cycloalkyl” intends a cyclic alkyl group, typically having 4 to 8, preferably 5 to 7, carbon atoms. The term “substituted alkyl” refers to alkyl groups substituted with one or more substituent groups, and include “heteroatom-containing alkyl” and “heteroalkyl,” which terms refer to alkyl groups in which at least one carbon atom is replaced with a heteroatom. If not otherwise indicated, the terms “alkyl” and “lower alkyl” include linear, branched, cyclic, unsubstituted, substituted, and/or heteroatom-containing alkyl and lower alkyl groups, respectively.

The term “alkylene” as used herein refers to a difunctional linear, branched, or cyclic alkyl group, where “alkyl” is as defined above.

The term “alkenyl” as used herein refers to a linear, branched, or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Preferred alkenyl groups herein contain 2 to about 12 carbon atoms. The term “lower alkenyl” intends an alkenyl group of 2 to 6 carbon atoms, and the specific term “cycloalkenyl” intends a cyclic alkenyl group, preferably having 5 to 8 carbon atoms. The term “substituted alkenyl” refers to alkenyl groups substituted with one or more substituent groups, and the terms “heteroatom-containing alkenyl” and “heteroalkenyl” refer to alkenyl groups in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl groups, respectively.

The term “alkenylene” as used herein refers to a difunctional linear, branched, or cyclic alkenyl group, where “alkenyl” is as defined above.

The term “alkynyl” as used herein refers to a linear or branched hydrocarbon group of 2 to about 24 carbon atoms containing at least one triple bond, such as ethynyl, n-propynyl, and the like. Preferred alkynyl groups herein contain 2 to about 12 carbon atoms. The term “lower alkynyl” intends an alkynyl group of 2 to 6 carbon atoms. The term “substituted alkynyl” refers to an alkynyl group substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include a linear, branched, unsubstituted, substituted, and/or heteroatom-containing alkynyl and lower alkynyl group, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms. Analogously, “alkenyloxy” and “lower alkenyloxy” respectively refer to an alkenyl and lower alkenyl group bound through a single, terminal ether linkage, and “alkynyloxy” and “lower alkynyloxy” respectively refer to an alkynyl and lower alkynyl group bound through a single, terminal ether linkage.

The term “aromatic” refers to the ring moieties which satisfy the Hückel 4n+2 rule for aromaticity, and includes both aryl (i.e., carbocyclic) and heteroaryl (also called heteroaromatic) structures, including aryl, aralkyl, alkaryl, heteroaryl, heteroaralkyl, or alk-heteroaryl moieties.

The term “aryl” as used herein, and unless otherwise specified, refers to an aromatic substituent or structure containing a single aromatic ring or multiple aromatic rings that are fused together, directly linked, or indirectly linked (such that the different aromatic rings are bound by a common group such as a methylene or ethylene moiety). Unless otherwise modified, the term “aryl” refers to carbocyclic structures. Preferred aryl groups contain 5 to 24 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituents in which at least one carbon atom is replaced with a heteroatom, as will be described in further detail infra.

The term “aryloxy” as used herein refers to an aryl group bound through a single, terminal ether linkage, wherein “aryl” is as defined above. An “aryloxy” group may be represented as —O-aryl where aryl is as defined above. Preferred aryloxy groups contain 5 to 24 carbon atoms, and particularly preferred aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxyphenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Preferred alkaryl and aralkyl groups contain 6 to 24 carbon atoms, and particularly preferred alkaryl and aralkyl groups contain 6 to 16 carbon atoms. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The terms “alkaryloxy” and “aralkyloxy” refer to substituents of the formula -OR wherein R is alkaryl or aralkyl, respectively, as just defined.

The term “acyl” refers to substituents having the formula —(CO)-alkyl, —(CO)-aryl, or —(CO)-aralkyl, and the term “acyloxy” refers to substituents having the formula —O(CO)-alkyl, —O(CO)-aryl, or —O(CO)-aralkyl, wherein “alkyl,” “aryl”, and “aralkyl” are as defined above.

The terms “cyclic” and “ring” refer to alicyclic or aromatic groups that may or may not be substituted and/or heteroatom-containing, and that may be monocyclic, bicyclic, or polycyclic. The term “alicyclic” is used in the conventional sense to refer to an aliphatic cyclic moiety, as opposed to an aromatic cyclic moiety, and may be monocyclic, bicyclic, or polycyclic. The term “acyclic” refers to a structure in which the double bond is not contained within a ring structure.

The terms “halo” and “halogen” are used in the conventional sense to refer to a chloro, bromo, fluoro, or iodo substituent.

The term “heteroatom-containing” as in a “heteroatom-containing group” refers to a hydrocarbon molecule or molecular fragment in which one or more carbon atoms is replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom containing, the terms “heteroaryl” and heteroaromatic” respectively refer to “aryl” and “aromatic” substituents that are heteroatom-containing, and the like. It should be noted that a “heterocyclic” group or compound may or may not be aromatic, and further that “heterocycles” may be monocyclic, bicyclic, or polycyclic as described above with respect to the term “aryl.” Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

By “substituted” as in “substituted alkyl”, “substituted aryl”, and the like, as alluded to in some of the aforementioned definitions, is meant that in the alkyl, aryl, or other moiety, at least one hydrogen atom bound to a carbon (or other) atom is replaced with one or more non-hydrogen substituents. Examples of such substituents include, without limitation: functional groups, such as halo, hydroxyl, sulfhydryl, C₁-C₂₄ alkoxy, C₂-C₂₄ alkenyloxy, C₂-C₂₄ alkynyloxy, C₅-C₂₄ aryloxy, C₆-C₂₄ aralkyloxy, C₆-C₂₄ alkaryloxy, acyl (including C₁-C₂₄ alkylcarbonyl (—CO-alkyl) and C₆-C₂₄ arylcarbonyl (—CO-aryl)), acyloxy (—O-acyl, including C₂-C₂₄ alkylcarbonyloxy (—O—CO-alkyl) and C₆-C₂₄ arylcarbonyloxy (—O—CO-aryl)), C₂-C₂₄ alkoxycarbonyl ((CO)—O-alkyl), C₆-C₂₄ aryloxycarbonyl (—(CO)—O-aryl), halocarbonyl (—CO)-X where X is halo), C₂-C₂₄ alkylcarbonato (—O—(CO)—O-alkyl), C₆-C₂₄ arylcarbonato (—O—(CO)—O-aryl), carboxy (—COOH), carboxylato (—COO—), carbamoyl (—(CO)NH₂), mono-(C₁-C₂₄ alkyl)-substituted carbamoyl (—(CO)NH(C₁-C₂₄ alkyl)), di-(C₁-C₂₄ alkyl)-substituted carbamoyl (—(CO)—N(C1-C₂₄ alkyl)2), mono-(C₁-C₂₄ haloalkyl)-substituted carbamoyl (—(CO)—NH(C₁-C₂₄ alkyl)), di-(C₁-C₂₄ haloalkyl)-substituted scarbamoyl (—(CO)—N(C₁-C₂₄ alkyl)₂), mono-(C₅-C₂₄ aryl)-substituted carbamoyl (—(CO)—NH-aryl), di-(C₅-C₂₄ aryl)substituted carbamoyl (—(CO)—N(C₅-C₂₄ aryl)₂), di-N—(C₁-C₂₄ alkyl),N—(C₅-C₂₄ aryl)-substituted carbamoyl, thiocarbamoyl —(C₅)—NH₂), mono-(C₁-C₂₄ alkyl)-substituted thiocarbamoyl (—(CO)—NH(C₁-C₂₄ alkyl)), di-(C₁-C₂₄ alkyl)-substituted thiocarbamoyl (—(CO)—N(C₁-C₂₄ alkyl)₂), mono-(C₅-C₂₄ aryl)substituted thiocarbamoyl (—(CO)—NH aryl), di-(C₅-C₂₄ aryl)-substituted thiocarbamoyl ((CO)—N(C₅-C₂₄ aryl)z), di-N-(C₁-C₂₄ alkyl), N-(C₅-C₂₄ aryl)-substituted thiocarbamoyl, carbamido (—NH—(CO)—NH₂), cyano(—C═N), cyanato (—O—C═N), thiocyanato (—S—C═N), formyl (—(CO)—H), thioformyl (—(CS)—H), amino (—NH₂), mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₁-C₂₄ alkylamido (—NH—(CO)-alkyl), C₆-C₂₄ arylamido (—NH—(CO)-aryl), imino (—CR═NH where R=hydrogen, C₁-C₂₄ alkyl, C₅-C₂₄ aryl, C₆-C₂₄ alkaryl, C₆-C₂₄ aralkyl, etc.), C₂-C₂₀ alkylimino (CR═N(alkyl), where R=hydrogen, C₁-C₂₄ alkyl, C₅-C₂₄ aryl, C₆-C₂₄ alkaryl, C₆-C₂₄ aralkyl, etc.), arylimino (—CR═N(aryl), where R=hydrogen, C₁-C₂₀ alkyl, C₅-C₂₄ aryl, C₆-C₂₄ alkaryl, C₆-C₂₄ aralkyl, etc.), nitro (—NO₂), nitroso (—NO), sulfo (—SO₂OH), sulfonate(SO₂O—), C₁-C₂₄ alkylsulfanyl (—S-alkyl; also termed “alkylthio”), C₅-C₂₄ arylsulfanyl (—S-aryl; also termed “arylthio”), C₁-C₂₄ alkylsulfinyl (—(SO)-alkyl), C₅-C₂₄ arylsulfinyl (—(SO)-aryl), C₁-C₂₄ alkylsulfonyl (—SO₂-alkyl), C₁-C₂₄ monoalkylaminosulfonyl-SO₂—N(H) alkyl), C₁-C₂₄ dialkylaminosulfonyl-SO₂—N(alkyl)₂, C₅-C₂₄ arylsulfonyl (—SO₂-aryl), boryl (—BH₂), borono (B(OH)₂), boronato (—B(OR)₂ where R is alkyl or aryl), phosphono (—P(O)(OH)₂), phosphonato (P(O)(O)₂), phosphinato (P(O)(O—)), phospho (—PO₂), and phosphine (—PH₂); and the moieties C₁-C₂₄ alkyl (preferably C₁-C₁₂ alkyl, more preferably C₁-C₆ alkyl), C₂-C₂₄ alkenyl (preferably C₂C₁₂ alkenyl, more preferably C₂-C₆ alkenyl), C₂-C₂₄ alkynyl (preferably C₂-C₁₂ alkynyl, more preferably C₂-C₆ alkynyl), C₅-C₂₄ aryl (preferably C₅-C₂₄ aryl), C₆-C₂₄ alkaryl (preferably C₆-C₁₆ alkaryl), and C₆-C₂₄ aralkyl (preferably C₆-C₁₆ aralkyl).

Where substituents are described as “substituted” or “optionally substituted,” these substitutions preferably comprise halo, hydroxyl, C₁-C₃ alkoxy, C₁-C₆ alkylcarbonyl (CO-alkyl), C₂-C₂₄ alkoxycarbonyl ((CO)—O-alkyl), carboxy (—COOH), carbamoyl (—(CO)—NH₂), mono-(C₁-C₆ alkyl)-substituted carbamoyl (—(CO)NH(C₁-C₆ alkyl)), di-(C₁-C₆ alkyl)-substituted carbamoyl (—(CO)—N(C₁-C₆ alkyl)₂), cyano(—C═N), cyanato (—O—C═N), thiocyanato (—S—C═N), formyl (—(CO)—H), amino (—NH₂), mono-(C₁-C₆ alkyl)-substituted amino, or di-(C₁-C₆ alkyl)substituted amino.

By “functionalized” as in “functionalized alkyl”, “functionalized olefin”, “functionalized cyclic olefin”, and the like, is meant that in the alkyl, olefin, cyclic olefin, or other moiety, at least one hydrogen atom bound to a carbon (or other) atom is replaced with one or more functional groups such as those described herein and above. The term “functional group” is meant to include any functional species that is suitable for the uses described herein. In particular, as used herein, a functional group would necessarily possess the ability to react with or bond to corresponding functional groups on a substrate surface.

In addition, the aforementioned functional groups may, if a particular group permits, be further substituted with one or more additional functional groups such as those specifically enumerated above. Analogously, the above-mentioned groups may be further substituted with one or more functional groups such as those specifically enumerated.

In a preferred embodiment of the present invention R is a C₂ or C₃ alkylene group which is unsubstituted or substituted with one or two, preferably two lower alkyl, preferably methyl or ethyl, more preferably methyl. Preferred examples for R are —CH₂—CH₂—, —CH₂—CH₂—CH₂—, —CH(CH₃)—CH(CH₃)—, —C(CH₃)₂—C(CH₃)₂— and —CH₂—C(CH₃)₂—CH₂—.

In the furfural derivative of Formula (II), R² independently is H, alkyl, alkenyl or aryl, as defined above. Preferably, R² independently is H or alkyl, more preferably H or C₁₋₄ alkyl, more preferably H or C₁₋₃ alkyl, even more preferably H or C₁₋₂ alkyl, most preferably H or methyl. In a further preferred embodiment, R² is H.

In the furfural derivative of Formula (II), R⁵ is R², —CH₂OR², —COR², —CO₂R² or

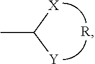

wherein X, Y and R and its preferred embodiments are defined as above.

Preferably, R⁵ is R², —CH₂OR², —COR², or —CO₂R², wherein R² is H or alkyl, wherein alkyl preferably is C₁₋₄ alkyl, in particular methyl or ethyl.

Certain embodiments of the furfural derivative of Formula (II) are the following compounds:

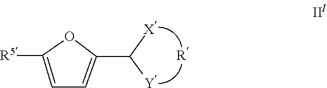

wherein R^{5′} is H, methyl, —CH₂OR^{2′}, —COR^{2′}, —CO₂R^{2′} or

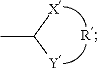

R^{2′} is H or alkyl, preferably H or C₁₋₄ alkyl;

X′ and Y′ are both O or S, or X′ is O and Y′ is —NR^{2′}— (preferably —NH— or —NCH₃—); and

R′ is C₂ or C₃ alkylene being optionally substituted with one, two or four C₁₋₄ alkyl (preferably methyl);

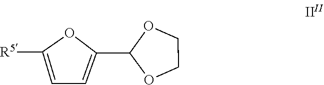

wherein R^{5′} is defined as above;

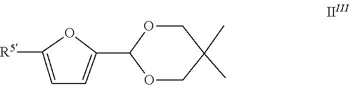

wherein R^{5′} is defined as above;

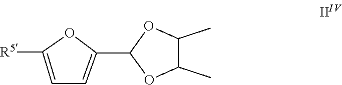

wherein R^{5′} is defined as above;

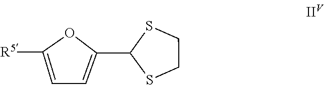

wherein R^{5′} is defined as above;

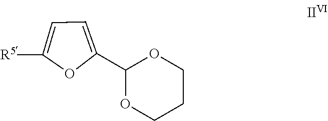

wherein R^{5′} is defined as above;

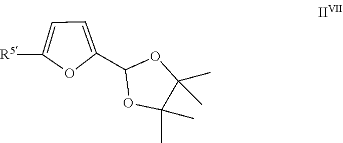

wherein R^{5′} is defined as above;

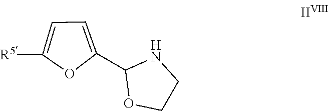

wherein R^{5′} is defined as above; and

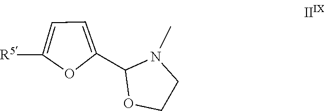

wherein R^{5′} is defined as above.

The furfural derivative of Formula (II) can be obtained for example by reacting furfural with ethylene glycol, substituted ethylene glycol or any other suitable dialcohol. This reaction, which also constitutes a protection of the aldehyde function of the furfural in the form of a cyclic ketal, is known to the person skilled in the art. The protection reaction can, for example, be carried out in a suitable organic solvent, such as cyclohexane, using a suitable catalyst, such as A70 Amberlyst® resin. For example, a furfural derivative of Formula (II), which is 1,3-dioxolan-2-(2-furanyl) can be obtained quantitatively by reacting furfural with ethylene glycol.

If compounds of Formula (II) containing hetero atoms other than oxygen are desired, furfural can be reacted for example with dithiols or amino alcohols.

According to the invention it has been found that the furfural derivative of Formula (II) reacts with an ethylene derivative of Formula (III) or (III′) thereby surprisingly resulting in the Diels-Alder adduct of Formula (I) having an increased meta/ortho ratio.

The ethylene derivative of Formula (III) or (III′) bears two substituents, R³ and R⁴. These substituents independently are H, —COR² or —CO₂R², provided that R³ and R⁴ are not identical. Thus, the ethylene derivative of Formula (III) or (III′) bears at least one substituent. In one embodiment, R⁴ is H and R³ is —COR² or —CO₂R². Alternatively, R³ is —COR² and R⁴ is —CO₂R².

In the compound of Formula (III) or (III′) R³ and R⁴ independently are H, —COR² or —CO₂R², wherein R² independently is H, alkyl, alkenyl or aryl. “Alkyl”, “alkenyl” and “aryl” preferably are as defined above. In a preferred embodiment R² is H or alkyl, preferably lower alkyl. In a further preferred embodiment R² is H or methyl.

Preferred compounds of Formula (III) are methyl vinyl ketone, methyl acrylate and acrolein.

The inventors have furthermore found that the meta/ortho ratio of the obtained Diels-Alder adduct is the highest if R³ or R⁴ is —COalkyl, in particular —COmethyl. A lower meta/ortho ratio is obtained if R³ or R⁴ is —CO₂alkyl, in particular —CO₂methyl. An even lower meta/ortho ratio is (which is, however, still higher than the meta/ortho ratio obtained with acrylonitrile) is obtained if R³ or R⁴ is —CHO. For example, it has been found that under the specific reaction conditions described in the Examples below at equilibrium the meta/ortho ratios were as follows: acrolein (63/37), methyl acrylate (67/33), and methyl vinyl ketone (87/13). Thus, if a high meta/ortho ratio is desired in the Diels-Alder adduct obtained by the process of the present invention, the compound of Formula (III) preferably is a compound wherein R³ or R⁴ is —COR², wherein R² is alkyl, alkenyl or aryl, more preferably wherein R³ or R⁴ is —COalkyl, even more preferably —COmethyl. Most preferably the compound of Formula (III) is methyl vinyl ketone.

The Diels-Alder condensation reaction between the compound of Formula (II) and the compound of Formula (III) or (III′) can be carried out under usual Diels-Alder conditions known to the person skilled in the art. Depending on the specific derivatives employed, the condensation reaction can be carried out in the presence or without any catalysts and also with or without any solvent. The reaction can be carried out at any suitable temperature of from about 10 to about 120° C., preferably from about 20 to about 100° C., more preferably from about 20 to about 80° C., for a time sufficient to convert the starting compounds into the desired Diels-Alder adduct, such as about 2 or 5 seconds to about 6 days, preferably about 3 hours to about 4 days, more preferably about 12 hours to about 4 days, such as about 24 hours. The reaction can be carried out at ambient pressure or increased pressure. Advantageously, the reaction is carried out at ambient pressure, such as about 1000 hPa or at a pressure of up to about 10000 hPa, preferably up to about 5000 hPa, more preferably up to about 2000 hPa.

Advantageously, the Diels-Alder reaction is conducted in the presence of a catalyst, in particular known Diels-Alder catalysts which may be supported on or provided by a solid material or a heterogeneous support, such as silica or a polymer. These catalysts include Lewis acids based on a metal, preferably a metal selected from the group consisting of Zn, Al, Sc, B, Fe, Ir, In, Hf, Sn, Ti, Yb, Sm, Cr, Co, Ni, Pb, Cu, Ag, Au, Tl, Hg, Pd, Cd, Pt, Rh, Ru, La, Ce, Pr, Nd, Pm, Eu, Gd, Tb, Dy, Ho, Er, Tm, Lu, V, Mn, Y, Zr, Nb, Mo, Ta, W, Re, Os and combinations thereof. More preferably, the catalyst is selected from the group consisting of Znl₂, ZnBr₂, ZnCl₂, Zn(Ac)₂, Sc(OSO₂CF₃)₃, Y(OSO₂CF₃)₃, Cu(OSO₂CF₃)₃, AlCl₃, Al(Et)₂Cl, Al(Et)Cl₂, BCl₃, BF₃, B(Ac)₃, FeCl₃, FeBr₃, FeCl₂, Fe(Ac)₂, Fe(Ac)₃, IrCl₃, HfCl₄, SnCl₄, TiCl₄, clays, zeolites and combinations thereof. Suitable Bronsted acids include inorganic mineral acids, e.g. sulfuric acid, phosphoric acid, nitric acid, hydrobromic acid or hydrochloric acid. Suitable organic acids include methane sulfonic acid, p-toluenesulfonic acid, or carboxylic acids. Diels-Alder catalysts also include halides of tin or titanium, such as SnCl₄ and TiCl₄. Alternatively, activated carbon, silica, alumina, silica-alumina, zirconia or zeolites may be used. Carbon, silica, alumina, silica-alumina, zirconia and zeolites may be used as such, but they may also be used as support for a catalytically active metal or metal compound. Such metals or metal compounds suitably include alkali metals, alkaline earth metals, transition metals, noble metals, rare earth metals. In a further alternative embodiment, the catalyst can be an organic compound, such as a proline derivative. The catalysts can be acidic, e.g. by treating supports with phosphoric acid, or by ion exchange of zeolites to render them into their acidic form. The catalyst can be an acid catalyst. Examples of solid catalysts include amorphous silica-alumina, zeolites, preferably zeolites in their H-form, and acidic ion exchange resins. Other suitable catalysts that are liquids or that may be dissolved in the appropriate solvent to yield a homogeneous catalyst environment, include organic and inorganic acids, such as alkane carboxylic acid, arene carboxylic acid, sulfuric acid, phosphoric acid, hydrochloric acid, hydrobromic acid and nitric acid.

The Diels-Alder condensation reaction between the compound of Formula (II) and the compound of Formula (III) or (III′) results in the oxanorbonene derivative of Formula (I). Depending on the starting compounds used, the obtained oxanorbonene may be obtained as different isomers (endo/exo and meta/ortho). All possible isomers are included within the scope of the present invention, although a meta/ortho ratio of above 1, preferably above 1.2, more preferably above 1.4, and even more preferably above 1.5 is preferred.

For example, if the compound of Formula (II) is reacted with acrolein, the resulting oxanorbonene derivative may be an ortho-isomer, a meta-isomer or a mixture of both. In other words, the oxanorbonene derivative can bear the —COH substituent resulting from the compound of Formula (III) either in ortho- or meta-position relative to the protected aldehyde substituent. Furthermore, both the meta-isomer and the ortho-isomer can be present as endo- or exo-isomer. Also these possible isomers are included within the scope of the present invention.

The various isomers can be distinguished from each other by NMR displacement determination.

The various isomers can be present as mixtures of two or more isomers or in the form of single isomers.

The oxanorbonene derivative of Formula (I) constitutes a valuable intermediate in the preparation of other chemical compounds, such as ortho-phthalaldehyde or isophthalaldehyde which in turn can be converted into ortho-xylenediamine or meta-xylenediamine (MXD) according to the following reaction scheme (showing a preferred example of the process according to the present invention):

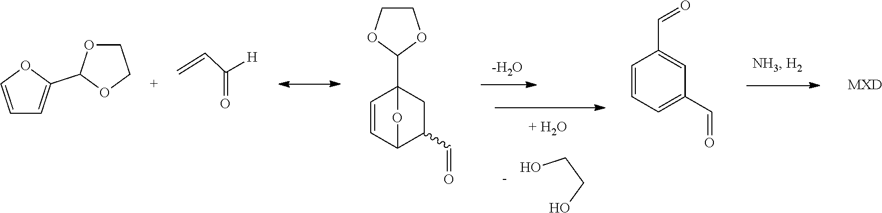

The aromatization and deprotection of the compound of Formula (I) can be carried out in a single step as described above. Alternatively, the desired compound of Formula (IV) can be obtained in a two-step process through the intermediate of the Formula (V). This alternative route is shown in the following reaction scheme which again exemplifies the reaction using preferred compounds:

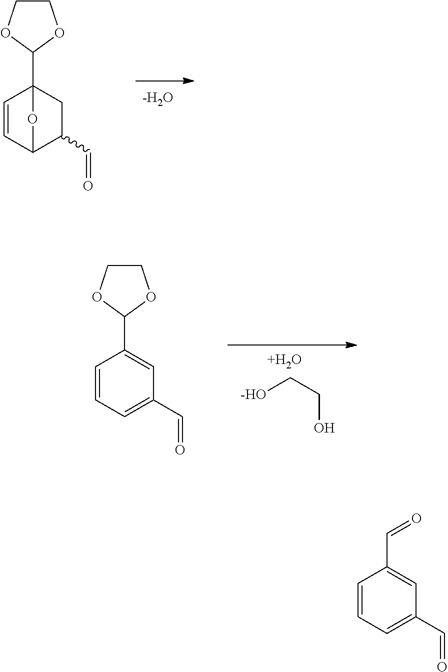

In an alternative embodiment, ortho-phthalaldehyde and isophthalaldehyde can be converted into their corresponding acids, namely phthalic acid and isophthalic acid, respectively:

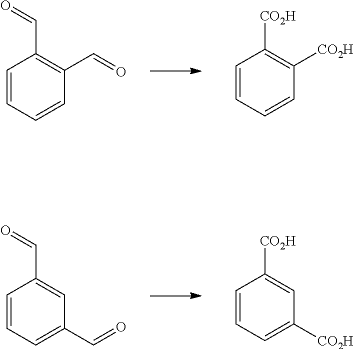 [SMILES: O=Cc1ccccc1C=O ; O=C(O)c1ccccc1C(=O)O ; O=Cc1cccc(C=O)c1 ; O=C(O)c1cccc(C(=O)O)c1]

In a further embodiment, the oxanorbonene derivative of formula (I) can be obtained by reacting the compound of formula (II) with a dienophile of formula (III) or (III′), wherein at least one of R³ and R⁴ (possibly, one and only one of R³ and R⁴) is —CO₂R². In this case, benzene derivatives being substituted with at least one aldehyde moiety and at least one carboxylic acid moiety (possibly, one and only one carboxylic acid moiety) can be obtained. In particular, notably when R⁵ is hydrogen, benzene derivatives being substituted with one and only one aldehyde moiety and at least one carboxylic acid moiety (possibly, one and only one carboxylic acid moiety) can be obtained. Also in this case, the aldehyde moiety can be further oxidized to a carboxylic acid moiety. This embodiment is exemplified by the following reaction scheme:

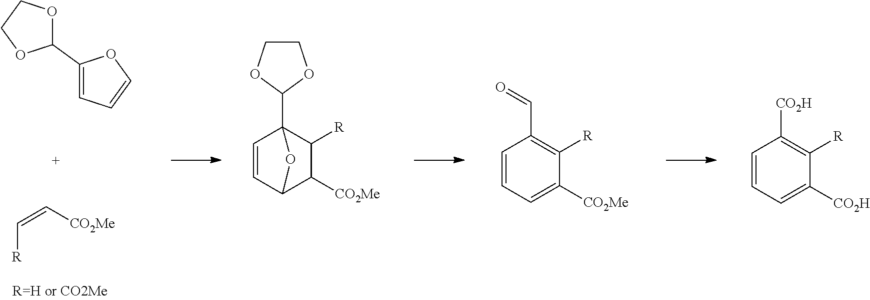

In a further embodiment the oxanorbonene derivative of formula (I) can bear two cyclic ketal substituents. In this case, the compound constitutes a valuable intermediate in the preparation of 1,4-substituted benzene derivatives, which may be further substituents in 2- and/or 3- position. Examples of such chemical compounds are 1,2,4-benzenetricarboxaldehyde and trimellitic acid. A possible route for the synthesis of these compounds is exemplified in the following reaction scheme:

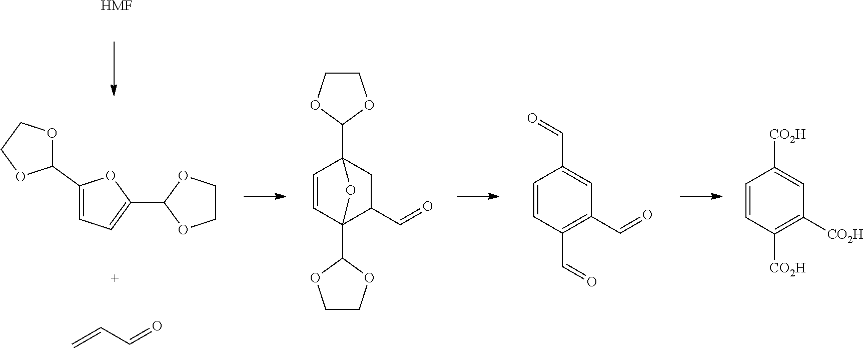

The present invention therefore also relates to a process for the preparation of a compound of Formula (IV)

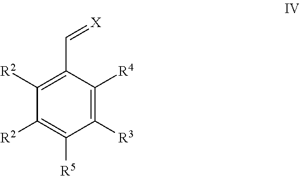

wherein
X is an optionally substituted heteroatom;
R² is independently H, alkyl, alkenyl or aryl;
R³ and R⁴ independently are H, —COR² or —CO₂R², provided that R³ and R⁴ are not identical; and
R⁵ is R², —CH₂OR², —COR² or —CO₂R² or

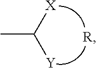

wherein Y is an optionally substituted heteroatom;
R is a C₁₋₄ alkylene group which may optionally be substituted with one or more R¹; and
R¹ is a linear or branched, saturated or unsaturated hydrocarbon group which optionally bears one or more functional groups;
a) which comprises dehydration/aromatization of a compound of the Formula (I)

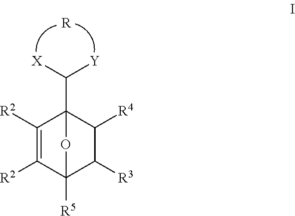

wherein X, Y, R, R², R³, R⁴ and R⁵ are defined as above;
to obtain a compound of the Formula (V)

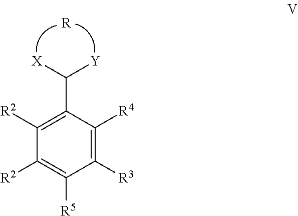

wherein X, Y, R, R², R³, R⁴ and R⁵ are defined as above;
followed by deprotection of the compound of Formula (V);
or
b) which comprises carrying out the dehydration/aromatization and the deprotection of the compound of Formula (I) in a single step.

The reaction conditions for aromatization and deprotection of the compound of Formula (I) are well known to a person skilled in the art. It was, however, surprisingly found that the aromatization reaction of the compound of Formula (I) requires basic reaction conditions, for example in the presence of a methoxide or hydroxide, such as sodium methoxide or sodium hydroxide. For example, the aromatization reaction can be conducted in quantitative yield using sodium methoxide in DMSO at a temperature of 100° C. for about 1 hour. Alcohols, such as methanol and ethanol are other suitable solvents.

Preferably, the compound of Formula (I) is obtained by the above described process using furfural and in particular the cyclic ketal derivative of furfural having the Formula (II) as starting material.

If desired, the compound of Formula (IV) obtained in the above process may be further converted into other chemical compounds, such as for example meta-xylenediamine, ortho-xylenediamine or 1,2,3-tri(aminomethyl)benzene. If meta-xylenediamine is the desired end product, the compound of Formula (IV) preferably is isophthalaldehyde.

Meta-xylenediamine can be obtained from isophthalaldehyde by reductive amination of the aldehyde moieties. Reductive amination can be conducted for example by reacting isophthalaldehyde in a solution of NH₃ in methanol (ratio NH₃/isophthalaldehyde about 19), at 100° C., 50 bar of hydrogen with Co Raney as catalyst.

Ortho-xylenediamine can be obtained from ortho-phthalaldehyde by reductive amination of the aldehyde moieties. Reductive amination can be conducted for example by reacting ortho-phthalaldehyde in a solution of NH₃ in methanol (ratio NH₃/ortho-phthalaldehyde about 19), at 100° C., 50 bar of hydrogen with Co Raney as catalyst.

1,2,3-Tri(aminomethyl)benzene can be obtained from benzene-1,2,3-tricarboxaldehyde by reductive amination of the aldehyde moieties. Reductive amination can be conducted for example by reacting benzene 1,2,3-tricarboxaldehyde in a solution of NH₃ in methanol (ratio NH₃/benzene-1,2,3-tricarboxaldehyde about 19) at 100° C., 50 bar of hydrogen with Co Raney as catalyst.

Thus, the present invention also relates to a process for the preparation of a xylene derivative of the Formula (VI)

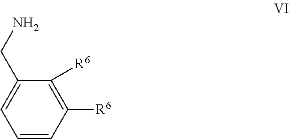

wherein R⁶ independently is H or —CH₂—NH₂, provided that at least one of R⁶ is —CH₂—NH₂;
which comprises:
preparing a compound of the Formula (VII)

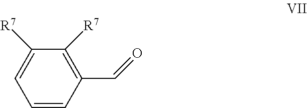

wherein R⁷ independently is H, —COR² or —CO₂R², provided that both R⁷ are not identical, and wherein R² independently is H, alkyl, alkenyl or aryl, and
proceeding with reductive amination of the compound of the Formula (VII).

Preferably, the compound of Formula (VII) is obtained by the above described processes.

In a further embodiment of the present invention, the process starting from the compounds of Formula (I) and (III) or (III′) until the compound of Formula (IV) is obtained can be carried out in a single step as one pot reaction.

The compounds of Formula (I) and Formula (V) are novel intermediates useful in the above described processes. Therefore, the present invention also relates to these compounds with the exception of compounds of Formula (I) wherein R is —CH₂—CH₂—, R² and R⁵ are H and R³ and R⁴ are —CO₂R² wherein R² in one case is H and in the other case is methyl (because these compounds are disclosed by S. Takano in Yakugaku Zasshi, 102 (2) 153-161 (1982)).

The oxanorbonene derivative of Formula (I) constitutes a valuable intermediate in the preparation of still other chemical compounds, such as orthophthalic acid, and isophthalic acid.

The present invention therefore also relates to the use of compounds of above formula (I) or above formula (II) for the manufacture of a benzene derivative, in particular a xylene derivative, trimethyl benzene derivative or tetramethyl benzene derivative. Preferred derivatives are ortho-phthalaldehyde, isophthalaldehyde, ortho-xylenediamine, meta-xylenediamine, 1,2,3-tri(aminomethyl)benzene, ortho-phthalic acid, isophthalic acid, trimellitic acid, 1,2,4-benzenetricarbaldehyde, 2-carbaldehydebenzoic acid, 3-carbaldehydeisophthalic acid and 2,3-dicarbaldehydebenzoic acid.

The present invention will now be illustrated by the following examples, which are not intended to be limiting.

### EXAMPLES

### Reference Example

### Synthesis of 2-(2-furyl)-1,3-dioxolane

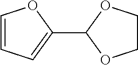

5.0 mL (60 mmol) of freshly distilled furfural, 10.0 mL (179 mmol, 3 eq) of ethylene glycol, 128.0 mg (0.6 mmol acid sites 0.01 eq) of Amberlyst® 15 resin and 60 mL (567 mmol) of toluene were charged in a single-neck round bottom flask equipped with magnetic stirring bar and Dean-Stark apparatus. The mixture was heated at 120° C. for 4 hours. The reaction mixture was cooled down and Amberlyst® 15 resin was filtered off. The reaction is quantitative. The 2-(2-furyl)-1,3-dioxolane was isolated as follow: 100 mL of ethyl acetate were added, and the organic phase was washed with water (20 mL, 3 times) to remove the excess of ethylene glycol. After drying over MgSO₄, ethyl acetate was evaporated under reduced pressure to afford 6.81 g of a colourless to pale yellow pure product (i.e. 81% isolated yield).

¹H NMR (400 MHz, DMSO-d₆) δ7.67 (dd, J=1.6, 0.8 Hz, 1H), 6.52 (dd, J=3.4, 0.8 Hz, 1H), 6.45 (dd, J=3.4, 1.6 Hz, 1H), 5.86 (s, 1H), 4.11-3.83 (m, 4H).

¹³C NMR (100 MHz, d6-DMSO) δ155.5, 143.4, 110.3, 108.9, 96.7, 64.5.

### Comparative Example

### Diels-Alder Reaction of 2-(2-furyl)-1,3-dioxolane with Acrylonitrile

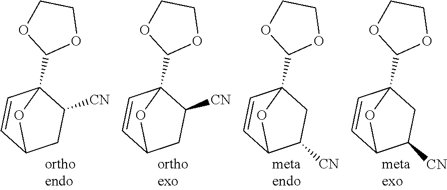

7.00 g (50 mmol) of 2-(2-furyl)-1,3-dioxolane and 16 mL (250 mmol, 5 eq) of acrylonitrile were charged in a carousel flask equipped with magnetic stirring bar and condenser. 681 mg (5 mmol, 0.1 eq) of zinc chloride were added as catalyst. The mixture was heated under nitrogen atmosphere at 60° C. for 25 hours. The reaction mixture was concentrated in vacuum. The recovered cycloadduct can be used as collected for subsequent aromatization reaction if the reaction is performed in absence of catalyst (longer time is required, up to 5 days). For characterization purpose, the cycloadduct was purified by flash chromatography (silica gel, EtOAc/cyclohexane) affording 6.8 g of cycloadducts (ortho/meta mixture) as yellow oil (i.e. 70% isolated yield).

### Ortho Endo

¹H NMR (400 MHz, DMSO-d6) δ6.66 (dd, J=5.8, 1.7 Hz, 1H), 6.46 (d, J=5.8 Hz, 1H), 5.28 (s, 1H), 5.14 (dd, J=4.6, 1.7 Hz, 1H), 4.11-3.83 (m, 4H), 3.11 (dd, J=9.4, 3.6 Hz, 1H), 2.37 (ddd, J=11.6, 9.4, 4.6 Hz, 1H), 1.52 (dd, J=11.6, 3.6 Hz, 1H).

¹³C NMR (100 MHz, DMSO-d₆) δ138.7, 132.2, 120.9, 101.0, 90.4, 78.8, 65.5, 65.2, 33.3, 26.1.

### Ortho Exo p ¹H NMR (400 MHz, DMSO-d₆) δ6.52 (dd, J=5.8, 1.6 Hz, 1H), 6.25 (d,

J=5.8 Hz, 1H), 5.32 (s, 1H), 5.18 (dd, J=4.6, 1.6 Hz, 1H), 4.11-3.83 (m, 4H), 2.75 (dd, J=8.3, 4.0 Hz, 1H), 2.02 (dt, J=11.6, 4.2 Hz, 1H), 1.86 (dd, J=11.6, 8.3 Hz, 1H).

¹³C NMR (100 MHz, DMSO-d₆) δ138.1, 132.2, 120.8, 101.0, 90.4, 78.3, 65.6, 65.2, 33.9, 29.0.

### Meta Endo

¹H NMR (400 MHz, DMSO-d₆) δ6.58 (d, J=5.8, 1.6 Hz, 1H), 6.54 (dd, J=5.8, 1.6 Hz, 1H), 5.31 (dd, J=4.4, 1.6 Hz, 1H), 5.23 (s, 1H), 4.11-3.83 (m, 4H), 3.30 (dt, J=9.5, 4.0 Hz, 1H), 2.20 (dd, J=11.4, 9.5 Hz, 1H), 1.41 (dd, J=11.4, 3.8 Hz, 1H).

¹³C NMR (100 MHz, DMSO-d₆) δ137.0, 133.9, 120.9, 101.1, 90.6, 78.8, 65.2, 65.1, 31.5, 27.3.

### Meta Exo

¹H NMR (400 MHz, DMSO-d₆) δ6.42 (d, J=5.8 Hz, 1H), 6.39 (dd, J=5.8, 1.6 Hz, 1H), 5.27 (s, 1H), 5.24 (d, J=1.6 Hz, 1H), 4.11-3.83 (m, 4H), 2.77 (dd, J=8.5, 3.9 Hz, 1H), 1.88 (dd, J=11.4, 3.9 Hz, 1H), 1.77 (dd, J=11.4, 8.5 Hz, 1H).

¹³C NMR (100 MHz, DMSO-d₆) δ136.2, 134.8, 122.6, 101.1, 89.9, 81.4, 65.2, 65.2, 31.6, 28.9.

### Example 1

### Diels-Alder reaction of 2-(2-furyl)-1,3-dioxolane with Methyl Vinyl Ketone

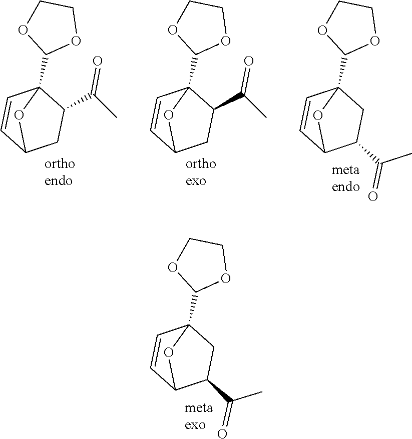

7.00 g (50 mmol) of 2-(2-furyl)-1,3-dioxolane and 21 mL (250 mmol, 5 eq) of methyl vinyl ketone were charged in a carousel flask equipped with magnetic stirring bar and condenser. The mixture was heated under nitrogen atmosphere at 60° C. for five days. The reaction mixture was concentrated in vacuum. The recovered cycloadduct can be used as collected for subsequent aromatization reaction. For characterization purpose, the cycloadduct was purified by flash chromatography (silica gel, EtOAc/cyclohexane) affording 3.2 g of cycloadducts (ortho/meta mixture) as yellow oil (i.e. 30% isolated yield).

### Ortho Endo

¹H NMR (400 MHz, DMSO-d₆) δ6.43 (dd, J=5.6, 1.6 Hz, 1H), 6.12 (d, J=5.6 Hz, 1H), 5.36 (s, 1H), 4.94 (dd, J=4.8, 1.6 Hz, 1H), 3.97-3.84 (m, 4H), 3.21 (dd, J=9.2, 4.4 Hz, 1H), 2.14 (m, 1H), 2.09 (s, 3H), 1.35 (dd, J=11.0, 4.4 Hz, 1H).

¹³C NMR (100 MHz, DMSO-d₆) δ207.3, 136.7, 132.0, 101.4, 90.6, 78.3, 65.0, 64.9, 50.3, 31.6, 30.7.

### Ortho Exo

¹H NMR (400 MHz, DMSO-d₆) δ6.45 (dd, J=5.6, 1.6 Hz, 1H), 6.28 (d, J=5.6 Hz, 1H), 5.19 (s, 1H), 5.09 (dd, J=4.8, 1.6 Hz, 1H), 3.97-3.84 (m, 4H), 2.56 (dd, J=8.2, 4.0 Hz, 1H), 2.15 (s, 3H), 1.94 (m, 1H), 1.50 (dd, J=11.4, 8.2 Hz, 1H).

¹³C NMR (100 MHz, DMSO-d₆) δ206.5, 137.2, 134.6, 100.8, 92.4, 78.1, 65.1, 65.0, 50.5, 32.2, 29.7.

### Meta Endo

¹H NMR (400 MHz, DMSO-d₆) δ6.35 (d, J=5.8 Hz, 1H), 6.24 (dd, 1.6Hz, 1H), 5.26 (dd, J=4.8, 1.6 Hz, 1H), 5.17 (s, 1H), 3.97-3.84 (m, 4H), 3.33 (m, 1H), 2.09 (s, 3H), 1.79 (dd, J=11.2, 9.2 Hz, 1H), 1.52 (dd, J=11.2, 4.0 Hz, 1H).

¹³C NMR (100 MHz, DMSO-d₆) δ205.5, 135.9, 132.8, 101.7, 90.5, 78.8, 65.0, 53.1, 29.3, 27.2.

### Meta Exo

¹H NMR (400 MHz, DMSO-d₆) δ6.45 (dd, J=6.0, 1.6 Hz, 1H), 6.33 (d, J=6.0 Hz, 1H), 5.16 (s, 1H), 5.12 (d, J=1.6 Hz, 1H), 3.97-3.84 (m, 4H), 2.58 (dd, J=8.4, 4.0, 1H), 2.17 (s, 3H), 1.91 (dd, J=11.2, 4.0 Hz, 1H), 1.41 (dd, J=11.2, 8.4 Hz, 1H).

¹³C NMR (100 MHz, DMSO-d₆) δ207.4, 136.0, 135.8, 101.8, 89.5, 79.9, 65.0, 52.4, 28.5, 28.2.

### Example 2

### Diels-Alder Reaction of 2-(2-furyl)-1,3-dioxolane with Methyl Acrylate

Diels-Alder reaction of 2-(2-furyl)-1,3-dioxolane with methyl acrylate was performed following the procedure previously described for the Diels-Alder reaction of 2-(2-furyl)-1,3-dioxolane with methyl vinyl ketone.

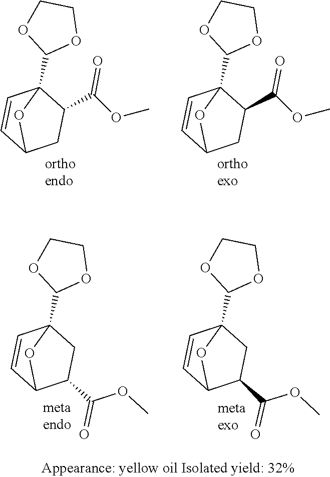

### Ortho Endo

¹H NMR (400 MHz, DMSO-d₆) δ6.49 (dd, J=5.6, 1.6 Hz, 1H), 6.17 (d, J=5.6 Hz, 1H), 5.50 (s, 1H), 4.98 (dd, J=4.8, 1.6 Hz, 1H), 3.99-3.83 (m, 4H), 3.55 (s, 3H), 3.01 (dd, J=9.6, 4.0 Hz, 1H), 2.17 (ddd, J=11.2, 9.6, 4.8, 1H), 1.42 (dd, J=11.2, 4.0 Hz, 1H).

NMR (100 MHz, DMSO-d₆) δ172.1, 137.2, 132.2, 101.0, 90.6, 78.4, 65.1, 65.0, 51.6, 42.1, 31.7.

### Ortho Exo

¹H NMR (400 MHz, DMSO-d₆) δ6.43 (dd, J=5.6, 1.6 Hz, 1H), 6.28 (d, J=5.6 Hz, 1H), 5.26 (s, 1H), 5.07 (dd, J=4.8, 1.6 Hz, 1H), 3.99-3.83 (m, 4H), 3.58 (s, 3H), 2.42 (dd, J=8.2, 4.0 Hz, 1H), 1.97 (m, 1H), 1.60 (dd, J=11.4, 8.2 Hz, 1H).

¹³C NMR (100 MHz, DMSO-d₆) δ173.1, 137.2, 134.6, 100.8, 91.7, 77.8, 65.0, 51.5, 42.9, 32.4.

### Meta Endo

¹H NMR (400 MHz, DMSO-d₆) δ6.41 (d, J=5.8 Hz, 1H), 6.26 (dd, J=5.8, 1.4 Hz, 1H), 5.18 (s, 1H), 5.12 (dd, J=5.0, 1.4 Hz, 1H), 3.99-3.83 (m, 4H), 3.56 (s, 3H), 3.26 (m, 1H), 1.96 (dd, J=11.2, 9.6 Hz, 1H), 1.47 (dd, J=11.2, 3.6 Hz, 1H).

¹³C NMR (100 MHz, DMSO-d₆) δ171.8, 136.3, 133.5, 101.6, 90.3, 78.7, 65.1, 51.6, 43.9, 28.7.

### Meta Exo

¹H NMR (400 MHz, DMSO-d₆) δ6.44 (dd, J=5.8, 1.6 Hz, 1H), 6.36 (d, J=5.8 Hz, 1H), 5.18 (s, 1H), 5.09 (d, J=1.6 Hz, 1H), 3.99-3.83 (m, 4H), 3.64 (s, 3H), 2.56 (dd, J=8.6, 4.0, 1H), 1.95 (dd, J=11.4, 4.0 Hz, 1H), 1.53 (dd, J=11.4, 8.6 Hz, 1H).

¹³C NMR (100 MHz, DMSO-d₆) δ173.3, 136.0, 135.7, 101.6, 89.5, 80.9, 65.0, 51.8, 44.2, 29.0.

### Example 3

### Diels-Alder Reaction of 2-(2-furyl)-1,3-dioxolane with Acrolein

Diels-Alder reaction of 2-(2-furyl)-1,3-dioxolane with acrolein was performed following the procedure previously described for the Diels-Alder reaction of 2-(2-furyl)-1,3-dioxolane with methyl vinyl ketone.

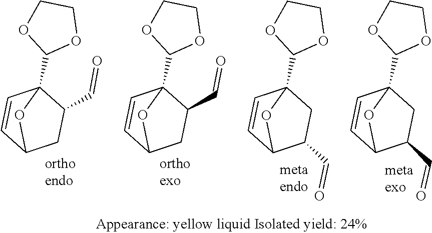

### Ortho Endo

¹H NMR (400 MHz, DMSO-d₆) δ9.27 (d, J=3.6 Hz, 1H), 6.57 (dd, J=6.0, 1.6 Hz, 1H), 6.28 (d, J=6.0 Hz, 1H), 5.28 (s, 1H), 5.04 (dd, J=4.6, 1.6 Hz, 1H), 4.03-3.76 (m, 4H), 3.00 (dt, J=9.0, 3.6 Hz, 1H), 2.06 (ddd, J=11.8, 9.0, 4.6 Hz, 1H), 1.53 (dd, J=11.8, 3.6 Hz, 1H).

¹³C NMR (100 MHz, DMSO-d₆) δ201.7, 138.5, 131.2, 101.5, 90.6, 78.8, 65.4, 65.1, 50.9, 28.9.

### Ortho Exo

¹H NMR (400 MHz, DMSO-d₆) δ9.35 (d, J=5.6 Hz, 1H), 6.54 (dd, J=6.0, 1.6 Hz, 1H), 6.30 (d, J=6.0 Hz, 1H), 5.29 (s, 1H), 5.16 (dd, J=4.8, 1.6 Hz, 1H), 4.03-3.76 (m, 4H), 2.28 (ddd, J=8.0, 5.6, 3.6 Hz, 1H), 2.04 (ddd, J=12.0, 4.8, 3.6 Hz, 1H), 1.51 (dd, J=12.0, 8.0 Hz, 1H).

¹³C NMR (100 MHz, DMSO-d₆) δ200.9, 138.4, 134.6, 100.9, 93.1, 78.5, 65.2, 64.8, 49.8, 28.5.

### Meta Endo

¹H NMR (400 MHz, DMSO-d₆) δ9.60 (d, J=2.4 Hz, 1H), 6.44 (dd, J=5.8, 1.6 Hz, 1H), 6.40 (d, J=5.8 Hz, 1H), 5.23 (dd, J=4.8, 1.6 Hz, 1H), 5.20 (s, 1H), 3.99-3.88 (m, 4H), 3.19 (m, 1H), 1.92 (dd, J=11.2, 9.0 Hz, 1H), 1.53 (dd, J=11.2, 4.0 Hz, 1H).

¹³C NMR (100 MHz, DMSO-d₆) δ202.0, 136.3, 133.2, 101.6, 90.5, 78.6, 65.1, 65.1, 52.9, 27.0.

### Meta Exo

¹H NMR (400 MHz, DMSO-d₆) δ9.35 (d, J=2.0 Hz, 1H), 6.41 (d, J=5.6 Hz, 1H), 6.34 (dd, J=5.6, 1.6 Hz, 1H), 5.24 (d, J=1.6 Hz, 1H), 5.21 (s, 1H), 3.99-3.88 (m, 4H), 2.51 (m, 1H), 1.97 (dd, J=11.6, 3.6 Hz, 1H), 1.43 (dd, J=11.6, 8.4 Hz, 1H).

¹³C NMR (100 MHz, DMSO-d₆) δ202.7, 136.4, 135.3, 101.7, 89.8, 79.1, 65.1, 65.1, 51.9, 26.9.

### Effect of the Dienophiles

The yields and meta/ortho ratios of the above examples and comparative example are summarized in the following table 1.

TABLE 1

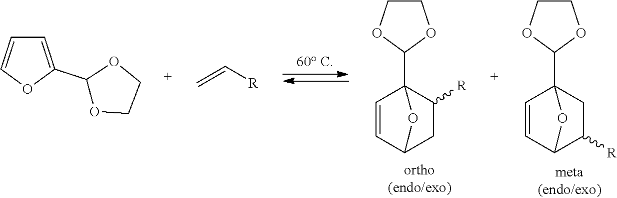

| | | DA cycloadduct | Yield (%) | % ortho | % meta |
|---|---|---|---|---|---|
| Example | R | formation rate | (at equilibrium) | (endo/exo) | (endo/exo) |
| Comparative Example | —CN | 0.08 | 76 | 48 (70/30) | 52 (60/40) |
| Example 1 | —COCH₃ | 0.18 | 36 | 13 (74/26) | 87 (65/35) |
| Example 2 | —COOCH₃ | 0.06 | 40 | 33 (87/13) | 67 (77/23) |
| Example 3 | —CHO | 0.40 | 28 | 38 (71/29) | 63 (43/57) |

From the data above, it is evident that the reaction with acrylonitrile (comparative example) results in a Diels-Alder adduct having a meta/ortho ratio of about 1. If the reaction is conducted with dienophiles according to the invention (examples 1, 2 and 3), the meta/ortho ratio is significantly increased.

1. A process for the preparation of a compound of Formula (I) 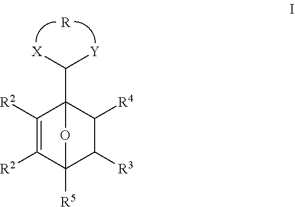
wherein
X and Y independently are optionally substituted heteroatoms;
R is a C₁₋₄ alkylene group which is optionally substituted with one or more R¹;
R¹ is a linear, branched and/or cyclic, saturated or unsaturated hydrocarbon group which optionally bears one or more functional groups;
R² independently is H, alkyl, alkenyl or aryl; and
R³ and R⁴ independently are H, —COR² or —CO₂R², provided that R³ and R⁴ are not identical; and 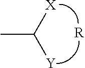
R⁵ is R², —CH₂OR², —COR², —CO₂R² or;
which comprises reacting a compound of Formula (II) 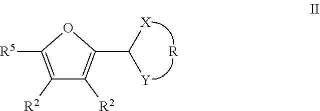
wherein X, Y, R, R² and R⁵ are defined as above;
with a compound of Formula (III) or (III′) 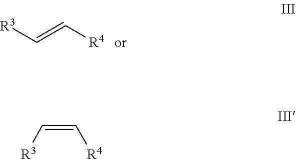
wherein R³ and R⁴ are defined as above.

2. The process according to claim 1, wherein
X and Y are O or X and Y are S, and
R is —CH₂—CH₂—, —CH₂—CH₂—CH₂—, —CH(CH₃)—CH(CH₃)—, —C(CH₃)₂—C(CH₃)₂— or —CH₂—C(CH₃)₂—CH₂—.

3. The process according to claim 1, wherein R² and R⁵ are H.

4. The process according to claim 1, wherein R³ or R⁴ is H.

5. A process for the preparation of a compound of Formula (IV) 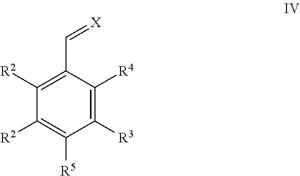
wherein
X is an optionally substituted heteroatom;
R² independently is H, alkyl, alkenyl or aryl;
R³ and R⁴ independently are H, —COR² or —CO₂R², provided that R³ and R⁴ are not identical;
R⁵ is R², —CH₂OR², —COR², —CO₂R² or 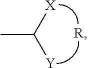
wherein Y is an optionally substituted heteroatom;
R is a C₁₋₄ alkylene group which is optionally substituted with one or more R¹; and
R¹ is a linear, branched and/or cyclic, saturated or unsaturated hydrocarbon group which optionally bears one or more functional groups;
a) which comprises dehydration/aromatization of a compound of Formula (I) 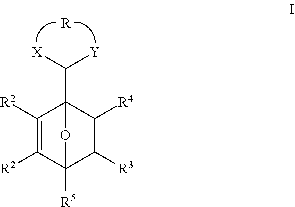
wherein X, Y, R, R², R³, R⁴ and R⁵ are defined as above;
to obtain a compound of Formula (V) 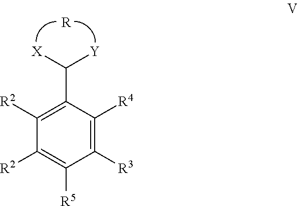
wherein X, Y, R, R², R³, R⁴ and R⁵ are defined as above;
followed by deprotection of the compound of Formula (V);
or
b) which comprises carrying out the dehydration/aromatization and the deprotection of the compound of Formula (I) in a single step.

6. The process according to claim 5, wherein:
X and Y are O or X and Y are S,
R is —CH₂—CH₂—, —CH₂—CH₂—CH₂—, —CH(CH₃)—CH(CH₃)—, —C(CH₃)₂—C(CH₃)₂— or —CH₂—C(CH₃)₂—CH₂—,
R² and R⁵ are H, and
R³ or R⁴ is H.

7. The process according to claim 5, which further comprises preparing the compound of Formula (I) by the process as defined in claim 1.

8. A process for the preparation of a xylene derivative of Formula (VI) 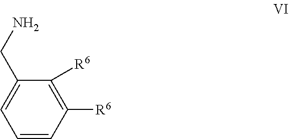
wherein R⁶ independently is H or —CH₂—NH₂, provided that at least one of R⁶ is —CH₂—NH₂;
which comprises:
preparing a compound of Formula (VII) 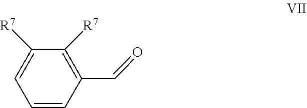
wherein R⁷ independently is H, —COR² or —CO₂R², provided that both R⁷ are not identical, and wherein R² independently is H, alkyl, alkenyl or aryl, and
proceeding with reductive amination of the compound of Formula (VII).

9. A compound of Formula (I) 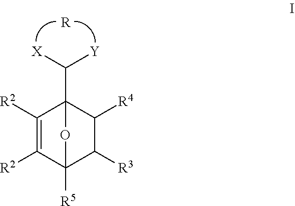
or of Formula (V) 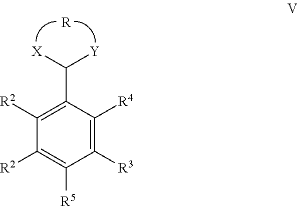
wherein
X and Y independently are optionally substituted heteroatoms;
R is a C₁₋₄ alkylene group which may optionally be substituted with one or more R¹;
R¹ is a linear, branched and/or cyclic, saturated or unsaturated hydrocarbon group which optionally bears one or more functional groups;
R² independently is H, alkyl, alkenyl or aryl;
R³ and R⁴ independently are H, —COR² or —CO₂R², provided that R³ and R⁴ are not identical; and 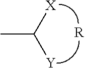
R⁵ is R², —CH₂OR², —COR², —CO₂R² or;
with the exception of compounds of Formula (I) wherein R is —CH₂—CH₂—, R² and R⁵ are H and R³ and R⁴ are —CO₂R² wherein R² in one case is H and in the other case is methyl.

10. The compound according to claim 9, wherein X and Y are O or X and Y are S.

11. The compound according to claim 9, wherein R² and R⁵ are H.

12. The compound according to claim 9, wherein R³ or R⁴ is H.

13. A process for manufacturing a benzene derivative, the process comprising:
manufacturing the benzene derivative from a compound of Formula (II) 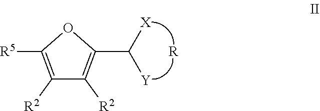
wherein
X and Y independently are optionally substituted heteroatoms;
R is a C₁₋₄ alkylene group which is optionally substituted with one or more R¹;
R¹ is a linear, branched and/or cyclic, saturated or unsaturated C₁₋₂₃ hydrocarbon group which optionally bears one or more functional groups;
R² independently is H, alkyl, alkenyl or aryl;
R³ and R⁴ independently are H, —COR² or —CO₂R², provided that R³ and R⁴ are not identical; and
R⁵ is R², —CH₂OR², —COR², —CO₂R² or

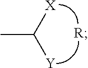

14. A process for manufacturing a benzene derivative, the method comprising manufacturing the benzene derivative from the compound of claim 9.

15. The process according to claim 13, wherein the benzene derivative is selected from the group consisting of ortho-phthalaldehyde, isophthalaldehyde, ortho-xylenediamine, meta-xylenediamine, 1,2,3-tri(aminomethyl)benzene, ortho-phthalic acid, isophthalic acid, trimellitic acid, 1,2,4-benzenetricarbaldehyde, 2-carb aldehydebenzoic acid, 3-carbaldehydeisophthalic acid, 2,3-dicarbaldehydebenzoic acid and combinations thereof.
